(19) United States
(12) Patent Application Publication
(10) Pub. No.: US 2021/0145369 A1
(43) Pub. Date: May 20, 2021

(54) APPARATUS AND METHOD FOR INDICATING THE NEURO-COGNITIVE AND PHYSIOLOGICAL CONDITION OF AN ANIMAL

(71) Applicant: Acrovirt, LLC, La Jolla, CA (US)

(72) Inventors: Terry Kennedy, La Jolla, CA (US); Steven de Belle, La Jolla, CA (US)

(73) Assignee: Acrovirt, LLC, La Jolla, CA (US)

(21) Appl. No.: 16/688,951

(22) Filed: Nov. 19, 2019

(51) Int. Cl. A61B 5/00; A61D 17/00; A61B 5/0205

(52) U.S. Cl. CPC A61B 5/7282; A61B 5/4812; A61B 5/4857; A61B 5/4875; A61B 5/04845; A61B 5/7267; A61D 17/002; A61B 5/0205; A61B 2503/40; A61B 5/4884

(57) ABSTRACT

Systems and methods are provided for indicating the neuro-cognitive and physiological condition of an animal. A system may comprise a hardware processor; and a non-transitory machine-readable storage medium encoded with instructions executable by the hardware processor to perform a method comprising: collecting physiological data representing one or more physiological indicators of an animal; determining a neuro-cognitive and physiological condition of the animal based on the one or more physiological indicators; and rendering the neuro-cognitive and physiological condition as a human-perceivable representation.

### DESCRIPTION OF RELATED ART

The disclosed technology relates generally to monitoring animals, and more particularly some embodiments relate to collecting and interpreting data from the animals and animal communication.

### BRIEF DESCRIPTION OF THE DRAWINGS

The present disclosure, in accordance with one or more various embodiments, is described in detail with reference to the following figures. The figures are provided for purposes of illustration only and merely depict typical or example embodiments.

FIG. 1 illustrates apparatus for collecting physiological indicators and determining neuro-cognitive and physiological conditions of a canine using one or more external EEG sensors, EKG sensors and other data collecting sensors near ears with non-invasive technology, data being shared through Bluetooth and processed through a mobile device with a voice processor for animal communication, according to some embodiments of the disclosed technology.

FIG. 2 illustrates an apparatus for collecting physiological indicators and determining neuro-cognitive and physiological conditions of a canine using one or more internal EEG sensors, EKG sensors and other data collecting sensors implanted under the skin and processed with a blue tooth collar and through a mobile device and voice box audio reproduction and/or translation of sound to words, comprehensible signals or images included on a mobile app for animal communication, according to some embodiments of the disclosed technology.

FIG. 3 illustrates apparatus for collecting physiological indicators and determining neuro-cognitive and physiological conditions of a feline using one or more external non-invasivore sensors, EKG sensors and other data collecting sensors above the skin and processed with, or without, a blue tooth collar and through a mobile device and voice box included on mobile app for animal communication, according to some embodiments of the disclosed technology.

FIG. 4 illustrates an apparatus for collecting physiological indicators and determining neuro-cognitive and physiological conditions of a feline using one or more internal EEG sensors, EKG sensors and other data collecting sensors implanted under the skin and processed with, or without, a blue tooth collar and through a mobile device and voice box included on mobile app for animal communication, according to some embodiments of the disclosed technology.

FIG. 5 illustrates apparatus for collecting physiological indicators and determining neuro-cognitive and physiological conditions of a rodent using one or more non-invasive external EEG sensors, EKG sensors and other data collecting sensors above the skin and processed with, or without, a blue tooth collar and through a mobile device and voice box included on mobile app for animal communication, according to some embodiments of the disclosed technology.

FIG. 6 illustrates an apparatus for collecting physiological indicators and determining neuro-cognitive and physiological conditions of a rodent using one or more internal EEG sensors, EKG sensors and other data collecting sensors implanted under the skin and processed with, or without, a blue tooth collar and through a mobile device and voice box included on mobile app for animal communication, according to some embodiments of the disclosed technology.

FIG. 7 shows detail of a monitoring system that may include blue tooth capability that communicates to a computer or mobile device, that may include one or more external EEG sensors, EKG sensors and other data collecting sensors, according to some embodiments of the disclosed technology.

FIG. 8 illustrates a block diagram of an apparatus for determining a neuro-cognitive and physiological condition of an animal according to some embodiments of the disclosed technology. (add voice box processor in app)

FIG. 9 shows detail of a monitoring system that may include one or more internal monitors 902 according to some embodiments of the disclosed technology.

FIG. 10 illustrates a block diagram of an apparatus for determining a neuro-cognitive condition of an animal according to some embodiments of the disclosed technology. (add voice box processor in app)

FIG. 11 illustrates a process for determining a neuro-cognitive and physiological condition of an animal according to some embodiments of the disclosed technology (through animal voice, audio sound cloning and reproduced into understandable words, comprehensible signals, images, or algorithmic score of processed data)

FIG. 12 illustrates a process for training a neural network for use in determining the neuro-cognitive and physiological condition of an animal according to embodiments of the disclosed technology.

FIG. 13 depicts a block diagram of an example computer system in which embodiments described herein may be implemented.

The figures are not exhaustive and do not limit the present disclosure to the precise form disclosed.

### DETAILED DESCRIPTION

Embodiments of the disclosure provide apparatus and methods for indicating the neuro-cognitive and physiological condition of an animal and allowing the animal to communicate in response to stimuli. The apparatus may include one or more sensors for collecting physiological indicators from the animal, and an analyzer for determining neuro-cognitive conditions of the animal based on the collected physiological indicators. For example, the physiological indicators collected by the sensors may include an electroencephalogram (EEG) of the animal. In this example, the analyzer may determine neuro-cognitive and physiological conditions of the animal based on the EEG, for example such as a level of comfort or agitation of the animal. But while some embodiments are described with reference to an EEG, it should be understood that any sensors, physiological indictors, and combinations thereof, may be used.

In some embodiments, the analyzer may determine neuro-cognitive and physiological conditions of the animal using a virtual library that stores relationships between physiological indicators and neuro-cognitive and physiological conditions. In some embodiments, the virtual library may be unique to the animal. In other embodiments, the virtual library may include data from many animals. In some embodiments, the virtual library may be implemented as a neural network or other network of presumptions. Additional examples are described in detail below.

In some embodiments, the animal is not human. The disclosed technology for determining neuro-cognitive and physiological conditions is useful for non-human animals because, not possessing the gift of language, non-human animals cannot describe their neuro-cognitive and physiological conditions. The non-human animals may include any non-human animal, for example including domestic animals, livestock, laboratory test animals, and the like. For a domestic animal, the disclosed technology may be useful for informing the owner when the animal is hungry, needs to go outside, and the like. The disclosed technology may allow an owner to determine whether an unusual movement of the animal is due to e.g., discomfort from injury, hunger, disease, aging or in response to living conditions. For livestock, the disclosed technology may be useful for determining the best time to feed the animal, the best time to gather milk or eggs, the best time to breed the animal, modification of rearing conditions, and the like. For laboratory test animals, the disclosed technology may be useful for collecting test data in a manner that is not harmful to the animal.

In some embodiments, the animal is human. In these embodiments, the disclosed technology may help determine a neuro-cognitive and physiological condition of the human when the human is unable to communicate effectively, when the human is given to prevarication, or for further research and development of neural-driven devices, and the like.

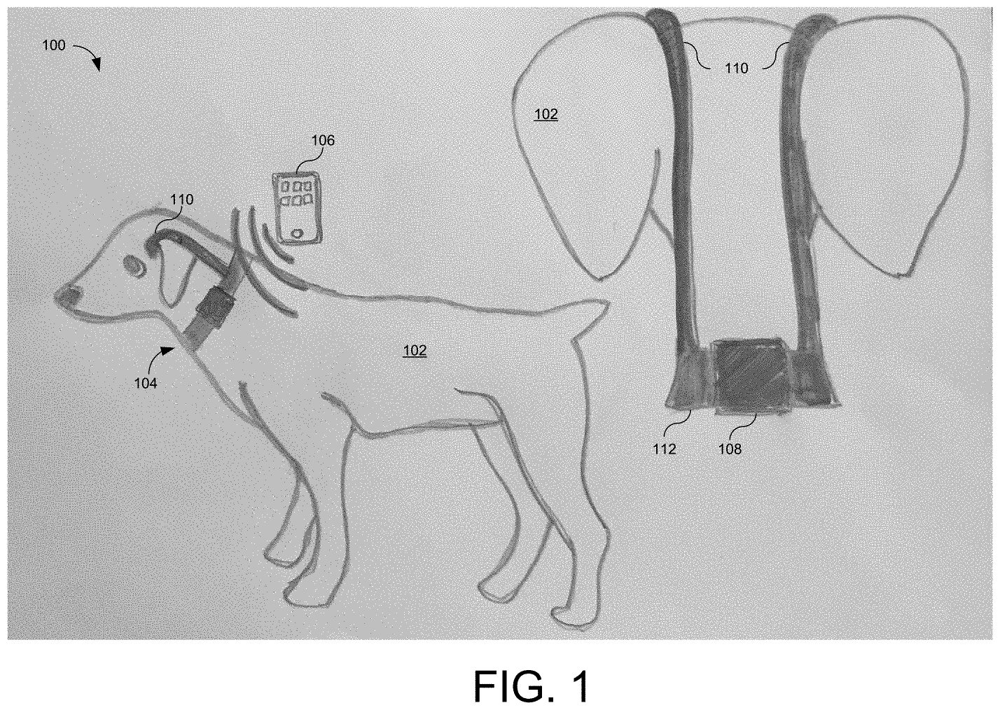

FIG. 1 illustrates apparatus 100 for determining neuro-cognitive and physiological conditions of a canine using one or more external EEG sensors according to some embodiments of the disclosed technology. Referring to FIG. 1, the apparatus 100 may include a monitoring device 104, which may be worn by a canine 102, and an analyzer 106 in communication with the monitoring device 104. The analyzer 106 may be implemented as a smart phone, computer, or the like. The analyzer 106 may communicate with the monitoring device 104 by wireless communications.

In the example of FIG. 1, the monitoring device 104 may include one or more EEG sensors 110. The monitoring device 104 may include structures to position the EEG sensors 110 near the temporal lobes of the brain of the canine 102. In some embodiments, the monitoring device 104 may include additional sensors. The monitoring device 104 may include a wireless transceiver 108 to support wireless communication with the analyzer 106. The monitoring device 104 may include an attachment device 112 to secure the monitoring device 104 to the canine 102. In the example of FIG. 1, the attachment device 112 is a dog collar.

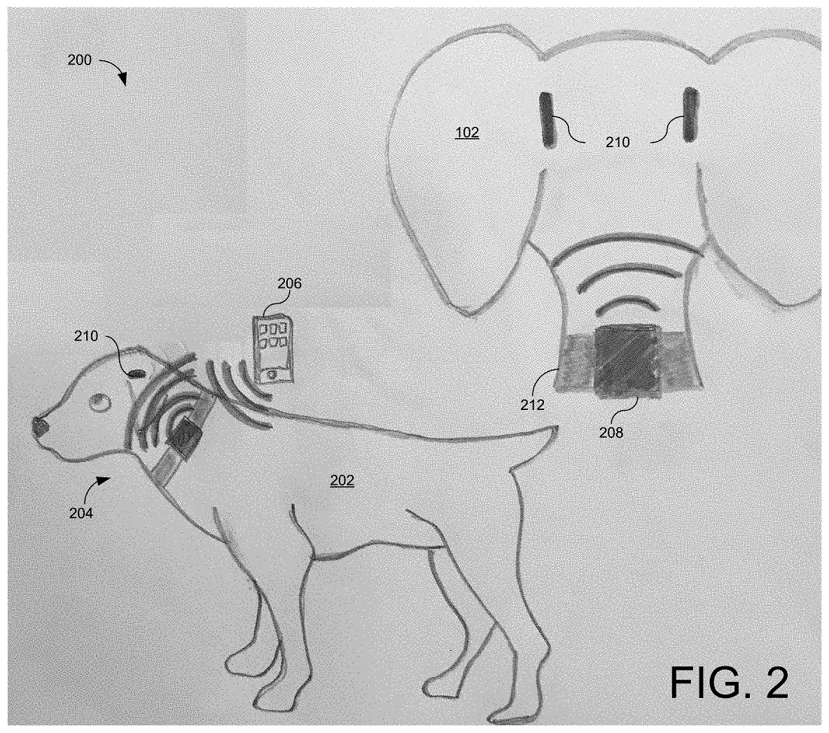

FIG. 2 illustrates an apparatus 200 for determining neuro-cognitive and physiological conditions of a canine using one or more internal EEG sensors according to some embodiments of the disclosed technology. Like the example of FIG. 1, the apparatus 200 may include a monitoring device 204, which may be worn by a canine 202, and an analyzer 206 in communication with the monitoring device 204. But unlike the example of FIG. 1, the apparatus may include one or more subcutaneous sensors 210, which may be inserted within or below the epidermis of the canine 202. The analyzer 206 may be implemented as a smart phone, computer, or the like.

In the example of FIG. 2, the sensors 210 may be implemented as EEG sensors, and may be placed near the temporal lobes of the brain of the canine 202. In some embodiments, the monitoring device 204 may include additional sensors. In some embodiments, the monitoring device 204 may relay data from the sensors 210 to the analyzer 206. In such embodiments, the monitoring device 204 may include a wireless receiver to receive wireless data from the sensors 210, and a wireless transmitter to transmit the sensor data to the analyzer 206. In other embodiments, the subcutaneous sensors 210 may communicate directly with the analyzer 206, for example by wireless communications.

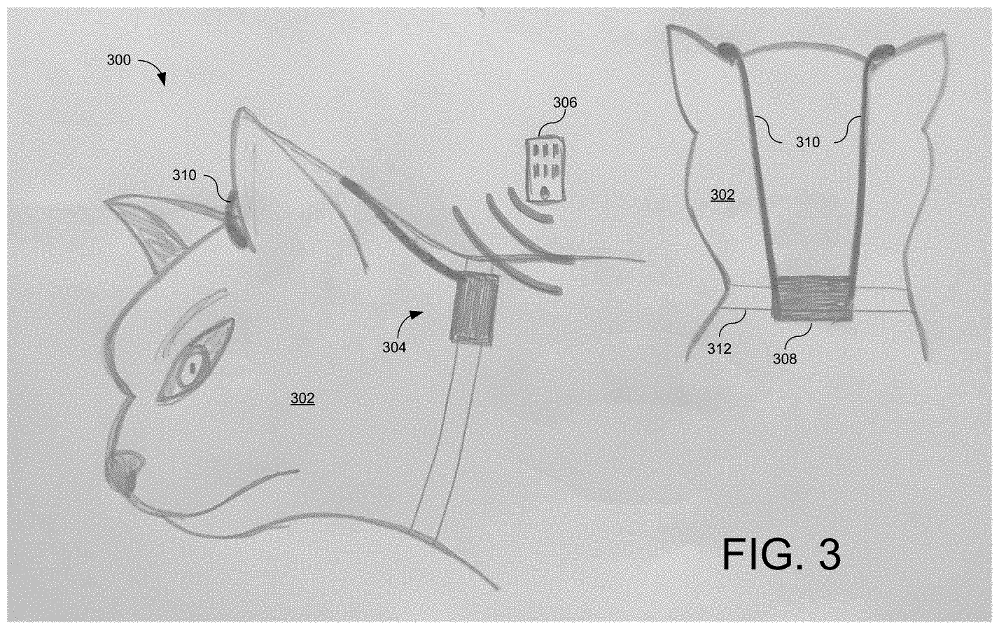

FIG. 3 illustrates apparatus 300 for determining neuro-cognitive and physiological conditions of a feline using one or more external EEG sensors according to some embodiments of the disclosed technology. The apparatus 300 may include a monitoring device 304, which may be worn by a feline 302, and an analyzer 306 in communication with the monitoring device 304. In the example of FIG. 3, the monitoring device 304 may include one or more EEG sensors 310. Elements of the apparatus 300 may operate similarly to corresponding elements of FIG. 1.

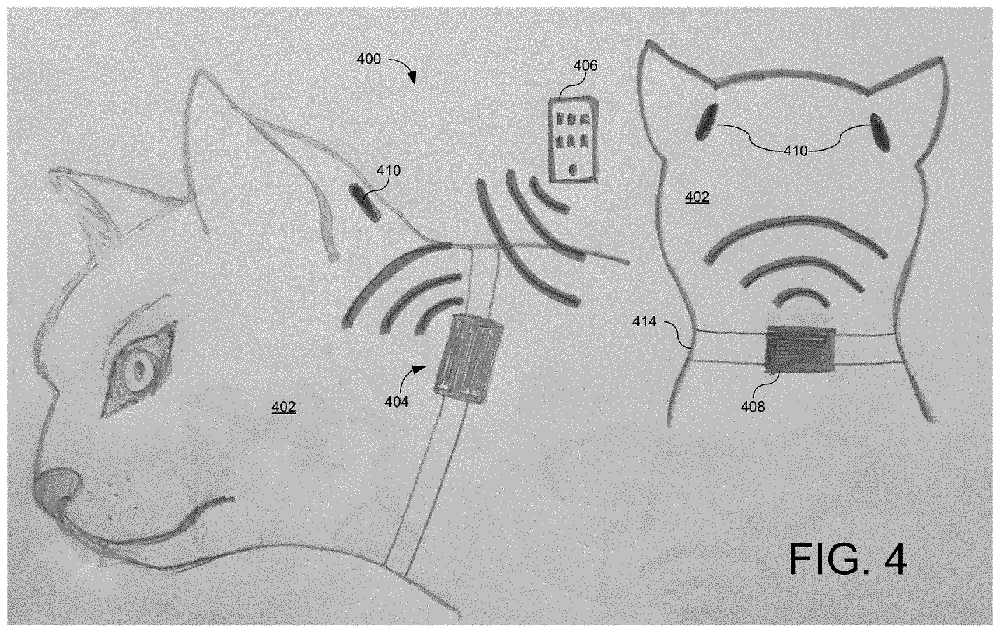

FIG. 4 illustrates an apparatus 400 for determining neuro-cognitive and physiological conditions of a feline using one or more internal EEG sensors according to some embodiments of the disclosed technology. The apparatus 400 may include a monitoring device 404, which may be worn by a feline 402, and an analyzer 406 in communication with the monitoring device 404. The apparatus 400 may include one or more subcutaneous EEG sensors 410. Elements of the apparatus 400 may operate similarly to corresponding elements of FIG. 2.

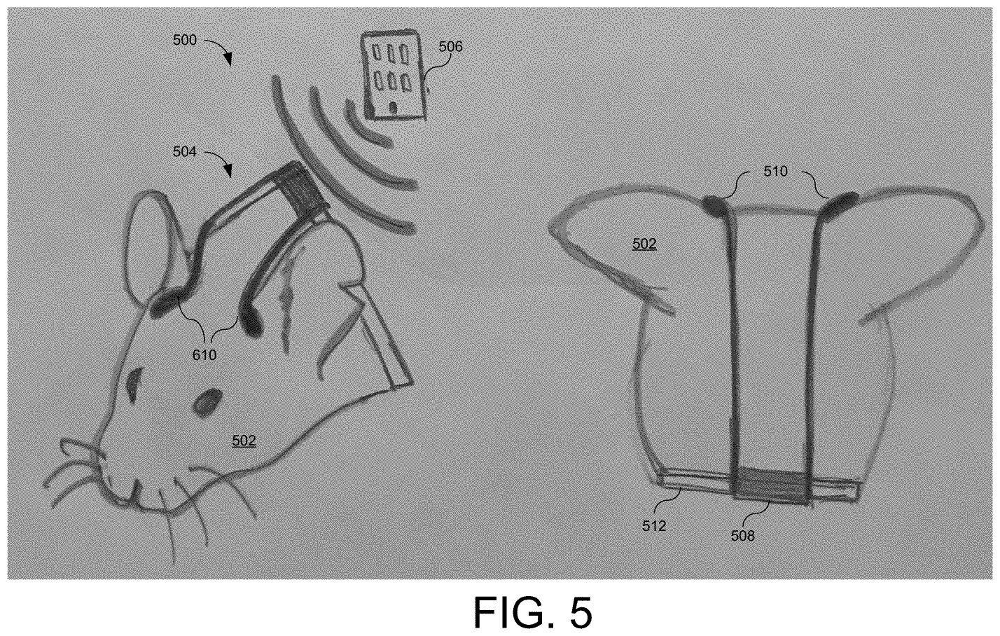

FIG. 5 illustrates an apparatus 500 for determining neuro-cognitive and physiological conditions of a rodent using one or more external EEG sensors according to some embodiments of the disclosed technology. The apparatus 500 may include a monitoring device 504, which may be worn by a rodent 502, and an analyzer 506 in communication with the monitoring device 504. In the example of FIG. 5, the monitoring device 504 may include one or more EEG sensors 510. Elements of the apparatus 500 may operate similarly to corresponding elements of FIG. 1.

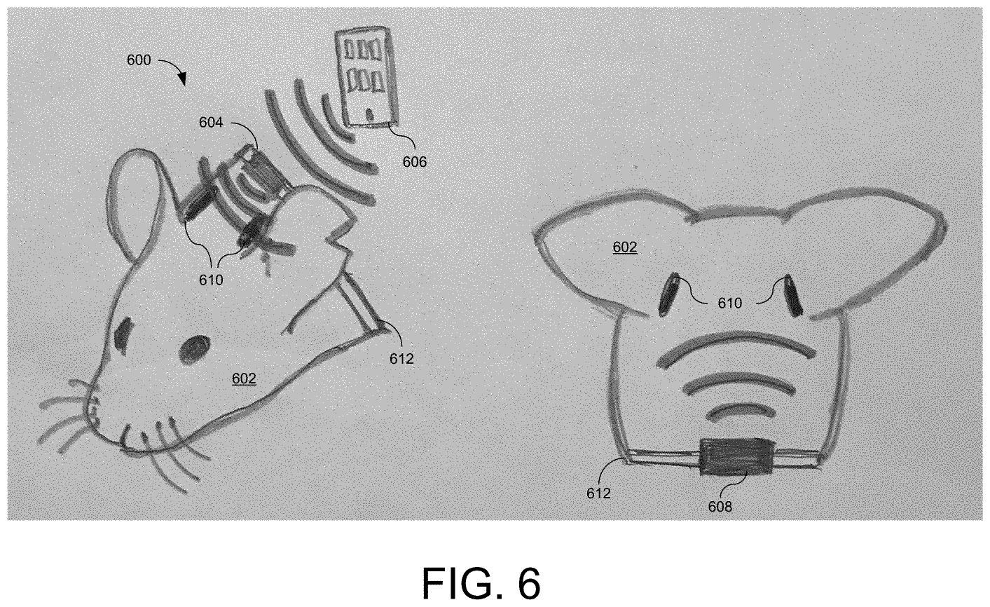

FIG. 6 illustrates an apparatus 600 for determining neuro-cognitive and physiological conditions of a rodent using one or more internal EEG sensors according to some embodiments of the disclosed technology. The apparatus 600 may include a monitoring device 604, which may be worn by a rodent 602, and an analyzer 606 in communication with the monitoring device 604. The apparatus 600 may include one or more subcutaneous EEG sensors 610. Elements of the apparatus 600 may operate similarly to corresponding elements of FIG. 2.

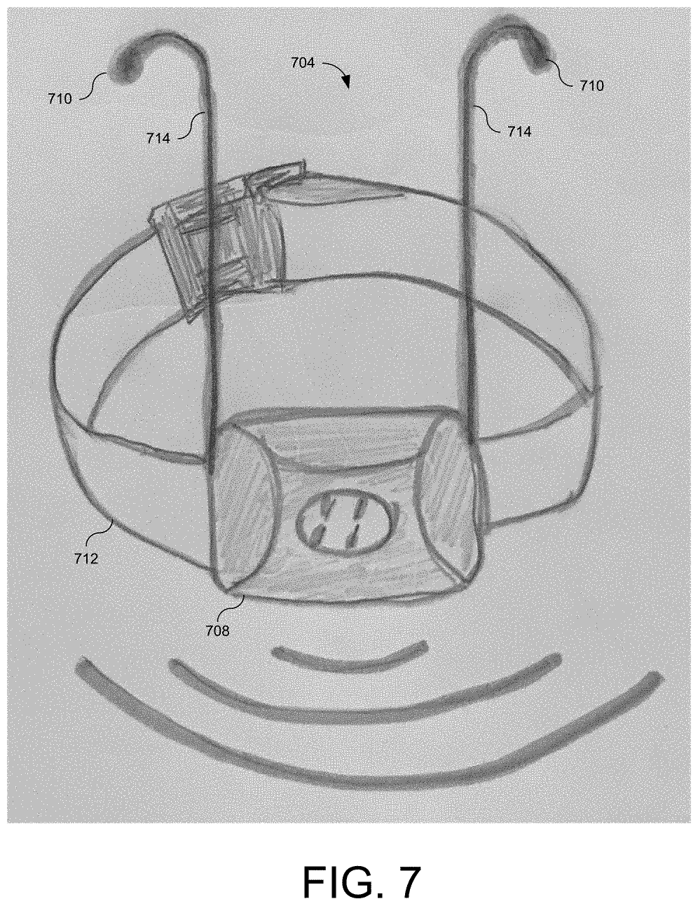

FIG. 7 shows detail of a monitoring system 700 that may include one or more external EEG sensors 710 according to some embodiments of the disclosed technology. Referring to FIG. 7, the monitoring system 700 may include a monitoring device 704. The monitoring device 704 may include one or more EEG sensors 710, and one or more stalks 714 to properly position the EEG sensors 710, for example near the temporal lobes of the brain of the animal being monitored. The monitoring device 704 may include a hub 708 in wired communication with the EEG sensors 710. The hub 708 may include a wireless transmitter for transmitting physiological indicators collected by the EEG sensors 710 to an analyzer. The hub 708 may include additional sensors as well. The monitoring device 704 may include an attachment device 712 for securing the hub 708, EEG sensors 710, and stalks 714 to the animal being monitored, for example as shown in FIGS. 1, 3, and 5.

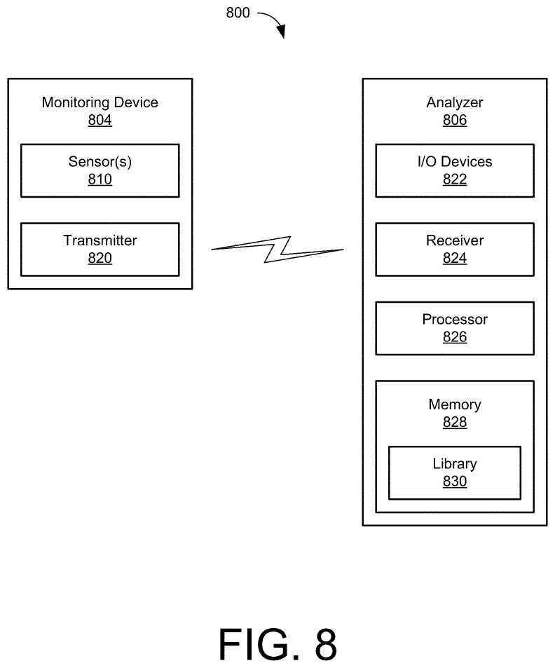

FIG. 8 illustrates a block diagram of an apparatus 800 for determining a neuro-cognitive and physiological condition of an animal according to some embodiments of the disclosed technology. The apparatus 800 may be used, for example, to implement the apparatus shown in FIGS. 1, 3, 5, and 7. Referring to FIG. 8, the apparatus 800 may include a monitoring device 804 and an analyzer 806. The monitoring device 804 may include one or more sensors 810 and a transmitter 820. The transmitter 820 may transmit wireless signals representing data collected by the sensors 802 to the analyzer 806. The sensors 810 may include any combination of sensors for collecting any combination of physiological indicators from an animal. For example, the sensors 802 may include one or more of an electroencephalography sensor an electrocardiography sensor, a blood pressure sensor, a thermometer, a saliva sensor, a motion sensor, a microphone, a camera, or any combination thereof. Other sensors may measure pupil dilation, eye tracking, and the like. The physiological indicators may include one or more of an electroencephalogram (EEG), an electrocardiogram (EKG), a heart rate, a blood pressure, saliva production, a speed, an acceleration, a sound, an image, or any combination thereof.

The analyzer 806 may include a wireless receiver 824 to receive the wireless signals transmitted by the transmitter 820 of the monitoring device 804. The analyzer 806 may include a processor 826 to process the received signals. The analyzer 806 may include a memory 828 to store the physiological indicator data collected from the monitoring device 804, and code executable by the processor 826 to perform the functions described herein. The memory 828 may also store a library 830. The library 830 may store relationships between physiological indicators and neuro-cognitive and physiological conditions, for example as described elsewhere herein. The analyzer 806 may include one or more input/output (I/O) devices 822 for controlling the analyzer 806, and for providing outputs to an operator of the analyzer 806.

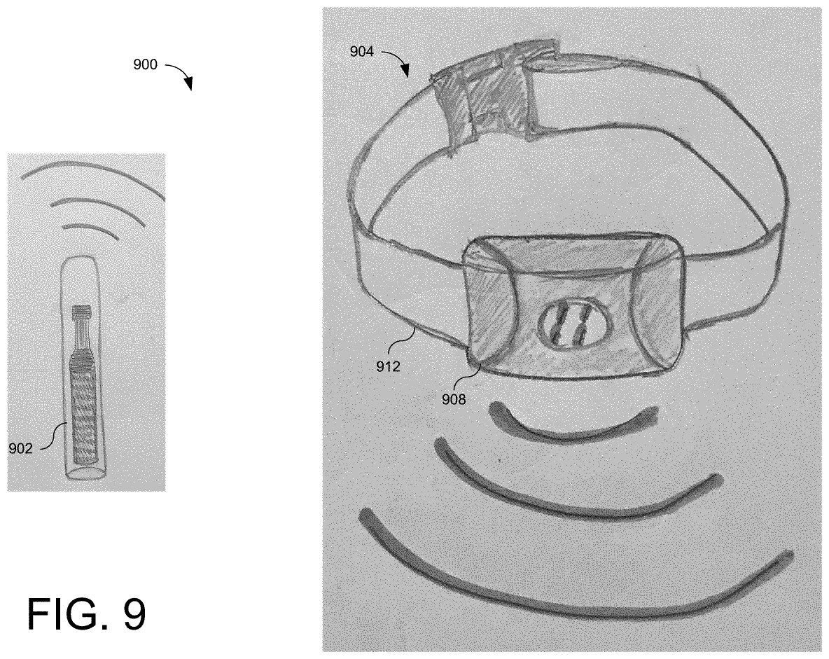

FIG. 9 shows detail of a monitoring system 900 that may include one or more internal monitors 902 according to some embodiments of the disclosed technology. Referring to FIG. 9, the system 900 may include a monitoring device 904 and one or more subcutaneous monitors 902, which may be located according to the type of data to be collected. For example, when a monitor 902 includes an EEG sensor, the monitor 902 may be located near the head and the brain of the animal being monitored. The monitor 902 may include one or more sensors, a microchip that includes a transmitter, a tuning capacitor, and a copper antenna coil, all of which may be encased in a biocompatible glass tube the size of an uncooked grain of rice.

The monitoring device 904 may include a hub 908 in wireless communication with the sensors 910. The hub 908 may include a wireless receiver for receiving physiological indicator data collected by the sensors 910, and a wireless transmitter for transmitting the physiological indicator data to an analyzer. The hub 908 may include additional sensors as well. The monitoring device 904 may include an attachment device 912 for securing the hub 908 to the animal being monitored, for example as shown in FIGS. 2, 4, and 6.

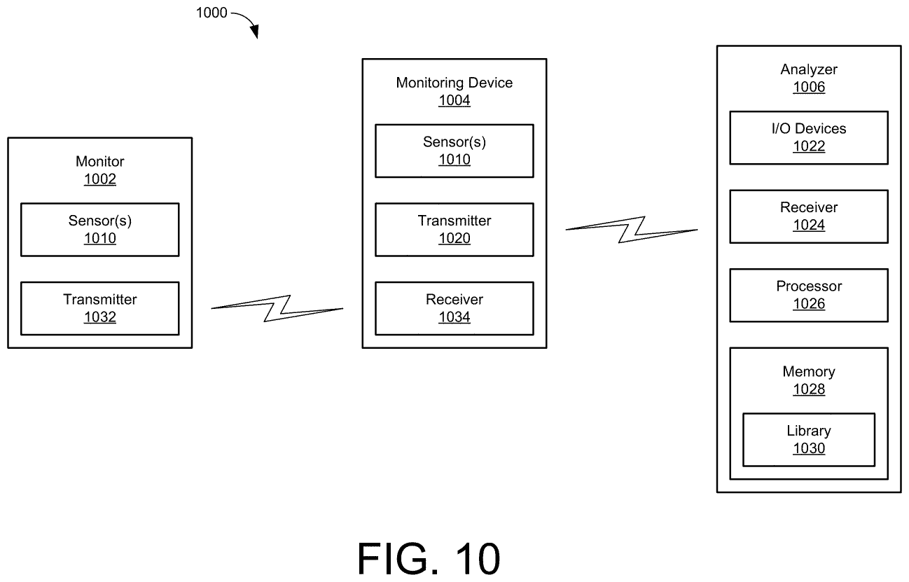

FIG. 10 illustrates a block diagram of an apparatus 1000 for determining a neuro-cognitive and physiological condition of an animal according to some embodiments of the disclosed technology. The apparatus 1000 may be used, for example, to implement the apparatus shown in FIGS. 2, 4, 6, and 9. Referring to FIG. 10, the apparatus 1000 may include a monitor 1002, a monitoring device 1004, and an analyzer 1006. The monitor 1002 may include one or more sensors 1010 and a transmitter 1032. The sensors 1010 may include any combination of sensors for collecting any combination of physiological indicators from an animal. The transmitter 1020 may transmit wireless signals representing data collected by the sensors 1010 to the monitoring device 1004. The monitoring device 1004 may include a receiver 1034 to receive the wireless signals transmitted by the monitor 1002. The monitoring device 1004 may include additional sensors 1010. The monitoring device 1004 may include a transmitter 1020 to transmit wireless signals representing data collected by the sensors 1010 to the analyzer 1006.

The analyzer 1006 may include a wireless receiver 1024 to receive the wireless signals transmitted by the transmitter 1020 of the monitoring device 1004. The analyzer 1006 may include a processor 1026 to process the received signals. The analyzer 1006 may include a memory 1028 to store the physiological indicators collected from the monitoring device 1004, and code executable by the processor 1026 to perform the functions described herein. The memory 828 may also store a library 1030. The library 1030 may store relationships between physiological indicators and neuro-cognitive and physiological conditions, for example as described elsewhere herein. The analyzer 1006 may include one or more input/output (I/O) devices 1022 for controlling the analyzer 1006, and for providing outputs to an operator of the analyzer 1006. In some embodiments, the analyzer 1006 may communicate directly with the monitor 1002.

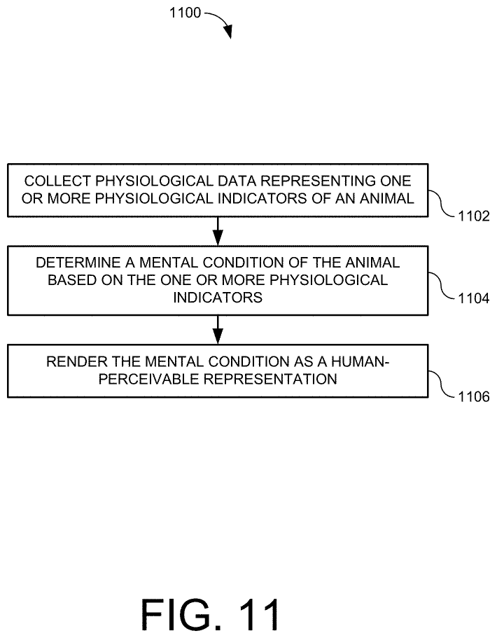

FIG. 11 illustrates a process 1100 for determining a neuro-cognitive and physiological condition of an animal according to some embodiments of the disclosed technology. Referring to FIG. 11, the process 1100 may include collecting physiological data representing one or more physiological indicators of an animal, at 1102. The physiological data may be collected by the sensors described in this disclosure and may represent the physiological indicators described herein. For example, the physiological data may include signals collected by EEG sensors from an animal. In this example, the physiological indicators may include an EEG of the animal. However, it should be appreciated that the physiological data and physiological indicators may include any physiological data and physiological indicators. For example, with livestock, the physiological data may include amounts of milk production. Other physiological data may indicate the position and movement of the animal, and the like. For aquatic animals, the physiological data may include electrical or pheromone signals in the water inhabited by the aquatic animals, such as possible prey or predator species. The animal may also generate audible sounds that may be collected. These sounds may be correlated with neural, physiological, behavioral, and situational data to give meaning (with some probability of accuracy) to these audible vocalizations.

The process 1100 may include determining a neuro-cognitive and physiological condition of the animal based on the one or more physiological indicators, at 1104. Continuing the EEG example, a neuro-cognitive and physiological condition of the animal may be determined based on the EEG of the animal. For example, the neuro-cognitive and physiological condition may include a level of comfort or agitation of the animal. In other examples, the neuro-cognitive and physiological condition of the animal may include one or more of an overall physiological state of the animal, a preference of the animal, an intention of the animal, and the like. The overall physiological state of the animal may include a temporal relationship to sleep, hibernation or biological rhythm (e.g., circadian, seasonal, etc.), temperature, humidity or other environmental parameter, a level of hunger or nutrient requirement, a reproductive status, a fitness of the animal, and the like. The level of agitation of the animal may be caused by one or more of a presence of one or more other animals having different social status than the monitored animal, a perceived threat, a deviation from a natural instinct for the animal, a deviation from a natural stimulus for the animal, and the like.

In some embodiments, neural network technology may be employed in determining the neuro-cognitive and physiological condition of the animal. For example, a neural network may be trained with data collected from the animal, with data collected from other animals, or a combination thereof. In embodiments that employ data collected from other animals, the data may be limited to data collected from animals that are similar in various aspects to the animal being monitored. For example, the training data may be limited to animals of the same species, breed, age, geographic location, and the like. In this way, individual animal differences can be compared with population means and variances to provide assessment tools for various applications. In some embodiments, the measurements of other animals may be used as population mean or “group normal” values for comparison and evaluative purposes. In some embodiments, the other animals maybe chosen to be similar in one or more aspects to “the subject” animal.

The process 1100 may include rendering the neuro-cognitive and physiological condition as a human-perceivable representation, at 1106. In the examples of FIGS. 8 and 10, the neuro-cognitive and physiological conditions may be rendered by the I/O devices 822 and 1022. For example, the neuro-cognitive and physiological condition of the animal may be rendered as a numerical representation, as an audio representation, as a video representation, and the like, or any combination thereof. A numerical representation may include, for example, numbers representing different neuro-cognitive and physiological conditions, and confidence values for one or more of the conditions. A video representation may include, for example, a multimedia presentation of one or more neuro-cognitive and physiological conditions. An audio representation may include, for example, an audible description of one or more neuro-cognitive and physiological conditions. For example, each neuro-cognitive and physiological condition may be assigned a suitable description. Responsive to determining a neuro-cognitive and physiological condition, the analyzer may read the corresponding aloud. For example, on determining a dog is hungry, the analyzer may announce “feed me!”.

In some embodiments, the process 1100 may operate in real time. That is, the collection of physiological data, determination of neuro-cognitive and physiological condition and rendering of the neuro-cognitive and physiological condition may all occur in real time. In such real-time embodiments, it is possible to conduct a form of communication with the animal. In some of these embodiments, the communication may be unilateral. That is, an observer may observe the neuro-cognitive and physiological conditions of the animal in real time. In others of these embodiments, the communication may be bilateral. For example, a user may issue commands to an animal that has been trained to respond to those commands, and the user may then observe the neuro-cognitive and physiological conditions of the animal that result from receiving those commands.

In some embodiments, the neuro-cognitive and physiological conditions of the animal collected in real time may be recorded and processed over a span of time to obtain temporal patterns of change in the animal. These patterns may be processed using operations including comparisons, integrations, and the like to reflect, qualify and quantify neuro-cognitive and physiological plasticity (i.e., learning and memory) in the animal.

As mentioned above, in some embodiments the neuro-cognitive and physiological condition of an animal may be determined using neural network technology. In such embodiments, the neural network is first trained in a training phase using data collected from one or more animals. Once the neural network has been trained, data collected from an animal may be applied to the neural network during an inference phase to determine a neuro-cognitive and physiological condition of the animal. In some embodiments, an automated training regime with an AI core may use the animal's neural signals to modify animal behavior and through a feedback mechanism, the neural network as well.

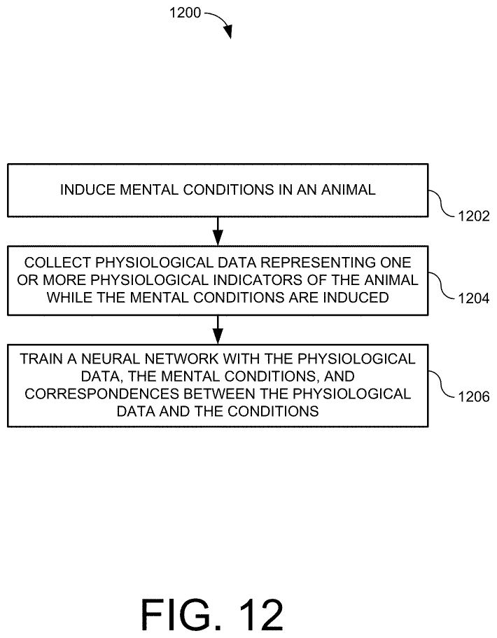

FIG. 12 illustrates a process 1200 for training a neural network for use in determining the neuro-cognitive and physiological condition of an animal according to embodiments of the disclosed technology. Referring to FIG. 12, the process 1200 may include inducing neuro-cognitive conditions in an animal, at 1202. Inducing a neuro-cognitive condition in an animal may include providing one or more stimuli to the animal. For example, the stimuli may include a physical stimulus, for example such as a mild electric shock. As another example, the stimuli may include giving the animal a command, where the animal has been trained to obey the command. Stimuli may include environ neuro-cognitive and physiological conditions, such as ambient air temperature, weather conditions, and the like. As another example, for fish the environ neuro-cognitive conditions may include water temperature, salinity, and mineral content, and the like. Other stimuli may be used instead of, or in addition to, these stimuli.

The process 1200 may include collecting physiological data representing one or more physiological indicators of the animal while the neuro-cognitive and physiological conditions are induced, at 1204. For example, physiological data may be collected from a dog while the dog is receiving a command the dog has been trained to obey.

The process 1200 may include training a neural network with the physiological data, the induced neuro-cognitive and physiological conditions, and correspondences between the physiological data in the conditions, at 1206. For example, continuing the example of the dog command, the neural network may be trained with the physiological data collected from the dog while the dog is receiving the command, and with the command as a label for the collected physiological data.

The disclosed technology has many applications. As described above, the disclosed technology may automatically verbalize a neuro-cognitive and physiological condition of an animal. As another example, determination of certain neuro-cognitive and physiological conditions of an animal may result in the operation of a device. For example, on determining a domestic animal would like to go outside, the analyzer could open an automatic pet door.

As another example, the disclosed technology may also associate determined neuro-cognitive and physiological conditions with a specific animal in a group of animals. In this example, each animal may be assigned a different “voice” so a user can identify the neuro-cognitive condition of a specific individual by recognizing the audible differences of the voice reporting the neuro-cognitive and physiological condition.

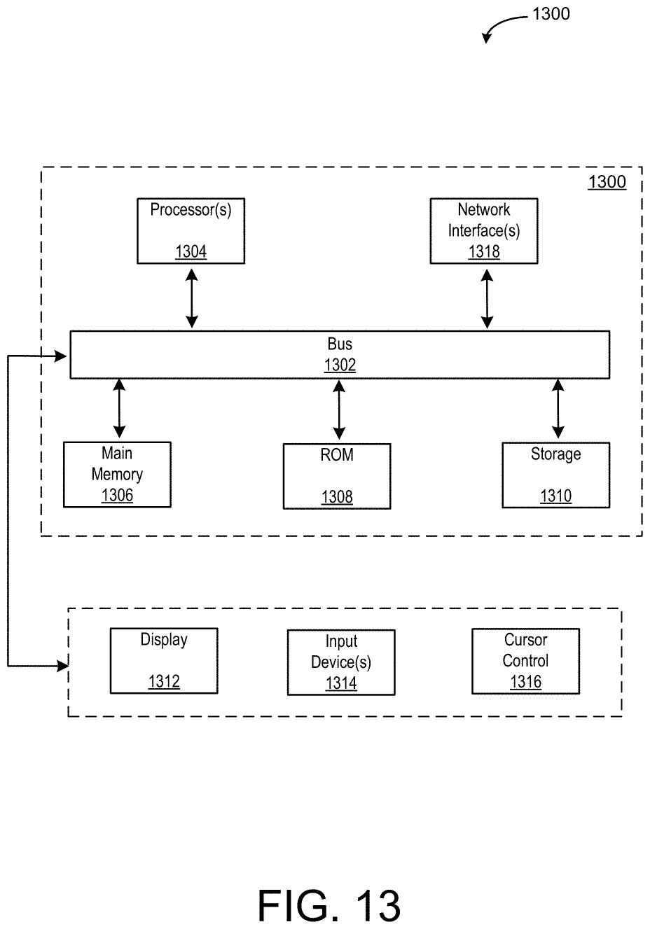

FIG. 13 depicts a block diagram of an example computer system 1300 in which embodiments described herein may be implemented. The computer system 1300 includes a bus 1302 or other communication mechanism for communicating information, and one or more hardware processors 1304 coupled with bus 1302 for processing information. Hardware processor(s) 1304 may be, for example, one or more general purpose microprocessors.

The computer system 1300 also includes a main memory 1306, such as a random-access memory (RAM), cache and/or other dynamic storage devices, coupled to bus 1302 for storing information and instructions to be executed by processor 1304. Main memory 1306 also may be used for storing temporary variables or other intermediate information during execution of instructions to be executed by processor 1304. Such instructions, when stored in storage media accessible to processor 1304, render computer system 1300 into a special-purpose machine that is customized to perform the operations specified in the instructions.

The computer system 1300 further includes a read only memory (ROM) 1308 or other static storage device coupled to bus 1302 for storing static information and instructions for processor 1304. A storage device 1310, such as a magnetic disk, optical disk, or USB thumb drive (Flash drive), etc., is provided and coupled to bus 1302 for storing information and instructions.

The computer system 1300 may be coupled via bus 1302 to a display 1312, such as a liquid crystal display (LCD) (or touch screen), for displaying information to a computer user. An input device 1314, including alphanumeric and other keys, is coupled to bus 1302 for communicating information and command selections to processor 1304. Another type of user input device is cursor control 1316, such as a mouse, a trackball, or cursor direction keys for communicating direction information and command selections to processor 1304 and for controlling cursor movement on display 1312. In some embodiments, the same direction information and command selections as cursor control may be implemented via receiving touches on a touch screen without a cursor.

The computing system 1300 may include a user interface module to implement a GUI that may be stored in a mass storage device as executable software codes that are executed by the computing device(s). This and other modules may include, by way of example, components, such as software components, object-oriented software components, class components and task components, processes, functions, attributes, procedures, subroutines, segments of program code, drivers, firmware, microcode, circuitry, data, databases, data structures, tables, arrays, and variables.

In general, the word “component”, “engine”, “system”, “database”, data store”, and the like, as used herein, can refer to logic embodied in hardware or firmware, or to a collection of software instructions, possibly having entry and exit points, written in an appropriate programming language. A software component may be compiled and linked into an executable program, installed in a dynamic link library, or may be written in an interpreted programming language. It will be appreciated that software components may be callable from other components or from themselves, and/or may be invoked in response to detected events or interrupts. Software components configured for execution on computing devices may be provided on a computer readable medium, such as a compact disc, digital video disc, flash drive, magnetic disc, or any other tangible medium, or as a digital download (and may be originally stored in a compressed or installable format that requires installation, decompression, or decryption prior to execution). Such software code may be stored, partially or fully, on a memory device of the executing computing device, for execution by the computing device. Software instructions may be embedded in firmware, such as an EPROM. It will be further appreciated that hardware components may be comprised of connected logic units, such as gates and flip-flops, and/or may be comprised of programmable units, such as programmable gate arrays or processors.

The computer system 1300 may implement the techniques described herein using customized hard-wired logic, one or more ASICs or FPGAs, firmware and/or program logic which, in combination with the computer system, causes or programs computer system 1300 to be a special-purpose machine. According to one embodiment, the techniques herein are performed by computer system 1300 in response to processor(s) 1304 executing one or more sequences of one or more instructions contained in main memory 1306. Such instructions may be read into main memory 1306 from another storage medium, such as storage device 1310. Execution of the sequences of instructions contained in main memory 1306 causes processor(s) 1304 to perform the process steps described herein. In alternative embodiments, hard-wired circuitry may be used in place of or in combination with software instructions.

The term “non-transitory media”, and similar terms, as used herein refers to any media that store data and/or instructions that cause a machine to operate in a specific fashion. Such non-transitory media may comprise non-volatile media and/or volatile media. Non-volatile media includes, for example, optical or magnetic disks, such as storage device 1310. Volatile media includes dynamic memory, such as main memory 1306. Common forms of non-transitory media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge, and networked versions of the same.

Non-transitory media is distinct from but may be used in conjunction with transmission media. Transmission media participates in transferring information between non-transitory media. For example, transmission media includes coaxial cables, copper wire, and fiber optics, including the wires that comprise bus 1302. Transmission media can also take the form of acoustic or light waves, such as those generated during radio-wave and infra-red data communications.

The computer system 1300 also includes a communication interface 1318 coupled to bus 1302. Network interface 1318 provides a two-way data communication coupling to one or more network links that are connected to one or more local networks. For example, communication interface 1318 may be an integrated services digital network (ISDN) card, cable modem, satellite modem, or a modem to provide a data communication connection to a corresponding type of telephone line. As another example, network interface 1318 may be a local area network (LAN) card to provide a data communication connection to a compatible LAN (or a WAN component to communicate with a WAN). Wireless links may also be implemented. In any such implementation, network interface 1318 sends and receives electrical, electromagnetic, or optical signals that carry digital data streams representing various types of information.

A network link typically provides data communication through one or more networks to other data devices. For example, a network link may provide a connection through local network to a host computer or to data equipment operated by an Internet Service Provider (ISP). The ISP in turn provides data communication services through the world wide packet data communication network now commonly referred to as the “Internet”. Local network and Internet both use electrical, electromagnetic, or optical signals that carry digital data streams. The signals through the various networks and the signals on the network link and through communication interface 1318, which carry the digital data to and from computer system 1300, are example forms of transmission media.

The computer system 1300 can send messages and receive data, including program code, through the network(s), network link, and communication interface 1318. In the Internet example, a server might transmit a requested code for an application program through the Internet, the ISP, the local network, and the communication interface 1318.

The received code may be executed by processor 1304 as it is received, and/or stored in storage device 1310, or other non-volatile storage for later execution.

Each of the processes, methods, and algorithms described in the preceding sections may be embodied in, and fully or partially automated by, code components executed by one or more computer systems or computer processors comprising computer hardware. The one or more computer systems or computer processors may also operate to support performance of the relevant operations in a “cloud computing” environment or as a “software as a service” (SaaS). The processes and algorithms may be implemented partially or wholly in application-specific circuitry. The various features and processes described above may be used independently of one another or may be combined in various ways. Different combinations and sub-combinations are intended to fall within the scope of this disclosure, and certain method or process blocks may be omitted in some implementations. The methods and processes described herein are also not limited to any particular sequence, and the blocks or states relating thereto can be performed in other sequences that are appropriate, or may be performed in parallel, or in some other manner. Blocks or states may be added to or removed from the disclosed example embodiments. The performance of certain of the operations or processes may be distributed among computer systems or computers processors, not only residing within a single machine, but deployed across a number of machines.

As used herein, a circuit might be implemented utilizing any form of hardware, or a combination of hardware and software. For example, one or more processors, controllers, ASICs, PLAs, PALs, CPLDs, FPGAs, logical components, software routines, or other mechanisms might be implemented to make up a circuit. In implementation, the various circuits described herein might be implemented as discrete circuits or the functions and features described can be shared in part or in total among one or more circuits. Even though various features or elements of functionality may be individually described or claimed as separate circuits, these features and functionality can be shared among one or more common circuits, and such description shall not require or imply that separate circuits are required to implement such features or functionality. Where a circuit is implemented in whole or in part using software, such software can be implemented to operate with a computing or processing system capable of carrying out the functionality described with respect thereto, such as computer system 1300.

As used herein, the term “or” may be construed in either an inclusive or exclusive sense. Moreover, the description of resources, operations, or structures in the singular shall not be read to exclude the plural. Conditional language, such as, among others, “can”, “could”, “might”, or “may”, unless specifically stated otherwise, or otherwise understood within the context as used, is generally intended to convey that certain embodiments include, while other embodiments do not include, certain features, elements and/or steps.

Terms and phrases used in this document, and variations thereof, unless otherwise expressly stated, should be construed as open ended as opposed to limiting. Adjectives such as “conventional”, “traditional”, “normal”, “standard”, “known” and terms of similar meaning should not be construed as limiting the item described to a given time period or to an item available as of a given time, but instead should be read to encompass conventional, traditional, normal, or standard technologies that may be available or known now or at any time in the future. The presence of broadening words and phrases such as “one or more”, “at least”, “but not limited to” or other like phrases in some instances shall not be read to mean that the narrower case is intended or required in instances where such broadening phrases may be absent.

What is claimed is:

1. A system, comprising:
a hardware processor; and
a non-transitory machine-readable storage medium encoded with instructions executable by the hardware processor to perform a method comprising:
collecting physiological data representing one or more physiological indicators of an animal;
determining a neuro-cognitive and physiological condition of the animal based on the one or more physiological indicators; and
rendering the neuro-cognitive and physiological condition as a human-perceivable representation.

2. The system of claim 1, wherein the neuro-cognitive and physiological condition of the animal comprises at least one of:
an overall physiological state of the animal;
a level of comfort of the animal;
a level of agitation of the animal;
a preference of the animal; and
an intention of the animal.

3. The system of claim 2, wherein the overall physiological state of the animal comprises at least one of:
temporal relationship to sleep, hibernation or biological rhythm;
level of hunger, hydration, or nutrient requirement;
reproductive status; and
fitness.

4. The system of claim 2, wherein the level of agitation of the animal is caused by at least one of:
a presence of one or more other animals having different social status than the animal;
a perceived threat or perceived target of the animal's possible aggression;
a deviation from a natural instinctive behavior of the animal; and
a deviation from a natural stimulus for the animal.

5. The system of claim 1, the method further comprising:
collecting the physiological data in real time; and
determining the neuro-cognitive and physiological condition of the animal in real time.

6. The system of claim 1, the method further comprising:
collecting the physiological data in real time; and
determining and comparing the neuro-cognitive and physiological condition of the animal in real time and over time, recording, comparing, and integrating temporal patterns of change in the animal that reflect, qualify, and quantify neuro-cognitive and physiological plasticity.

7. The system of claim 1, wherein determining the neuro-cognitive and physiological condition of the animal comprises:
applying the physiological data to a neural network, wherein the neural network has been trained with training data, the training data representing physiological indicators of a plurality of animals and corresponding neuro-cognitive and physiological conditions of the plurality of animals.

8. The system of claim 1, wherein collecting the physiological data representing one or more physiological indicators of the animal comprises:
collecting one or more signals each generated by one or more respective sensors disposed on or inside the animal.

9. A non-transitory machine-readable storage medium encoded with instructions executable by a hardware processor of a computing component, the machine-readable storage medium comprising instructions to cause the hardware processor to perform a method comprising:
collecting physiological data representing one or more physiological indicators of an animal;
determining a neuro-cognitive and physiological condition of the animal based on the one or more physiological indicators; and
rendering the neuro-cognitive and physiological condition as a human-perceivable representation.

10. The medium of claim 9, wherein the neuro-cognitive and physiological condition of the animal comprises at least one of:
an overall physiological state of the animal;
a level of comfort of the animal;
a level of agitation of the animal;
a preference of the animal; and
an intention of the animal.

11. The medium of claim 10, wherein the overall physiological state of the animal comprises at least one of:
temporal relationship to sleep, hibernation or biological rhythm;
level of hunger, hydration, or nutrient requirement;
reproductive status; and
fitness.

12. The medium of claim 10, wherein the level of agitation of the animal is caused by at least one of:
a presence of one or more other animals having different social status than the animal;
a perceived threat or perceived target of the animal's possible aggression;
a deviation from a natural instinctive behavior of the animal; and
a deviation from a natural stimulus for the animal.

13. The medium of claim 9, the method further comprising:
collecting the physiological data in real time; and
determining the neuro-cognitive and physiological condition of the animal in real time.

14. The medium of claim 9, the method further comprising:
collecting the physiological data in real time; and
determining and comparing the neuro-cognitive and physiological condition of the animal in real time and over time, recording, comparing, and integrating temporal patterns of change in the animal that reflect, qualify, and quantify neuro-cognitive and physiological plasticity.

15. The medium of claim 9, wherein determining the neuro-cognitive and physiological condition of the animal comprises:
applying the physiological data to a neuro network, wherein the neural network has been trained with training data, the training data representing physiological indicators of a plurality of animals and corresponding neuro-cognitive and physiological conditions of the plurality of animals.

16. The medium of claim 9, wherein collecting the physiological data representing one or more physiological indicators of the animal comprises:
collecting one or more signals each generated by one or more respective sensors disposed on or inside the animal.

17. A non-transitory machine-readable storage medium encoded with instructions executable by a hardware processor of a computing component, the machine-readable storage medium comprising instructions to cause the hardware processor to perform a method comprising:
inducing neuro-cognitive and physiological conditions in an animal;
collecting physiological data representing one or more physiological indicators of the animal while the neuro-cognitive and physiological conditions are induced; and
training a neural network with the physiological data, the neuro-cognitive and physiological conditions, and correspondences between the physiological data and the conditions.

18. The medium of claim 17, wherein the neuro-cognitive and physiological conditions comprise at least one of:
an overall physiological state of the animal;
a level of comfort of the animal;
a level of agitation of the animal;
a preference of the animal; and
an intention of the animal.

19. The medium of claim 18, wherein the overall physiological state of the animal comprises at least one of:
temporal relationship to sleep, hibernation or biological rhythm;
level of hunger;
reproductive status; and
fitness.

20. The medium of claim 18, wherein inducing the neuro-cognitive and physiological conditions in the animal comprises:
giving the animal a command, wherein the animal has been trained to obey the command.

21. The medium of claim 17, wherein inducing the neuro-cognitive and physiological conditions in the animal comprises:
providing a physical stimulus to the animal.

22. The medium of claim 17, the method further comprising:
collecting the physiological data in real time; and
determining and comparing the neuro-cognitive and physiological condition of the animal in real time and over time, recording, comparing, and integrating temporal patterns of change in the animal that reflect, qualify, and quantify neuro-cognitive and physiological plasticity.

23. The medium of claim 17, wherein collecting the physiological data comprises:
collecting one or more signals each generated by one or more respective sensors disposed on or inside the animal.
